(19) United States
(12) Patent Application Publication
(10) Pub. No.: US 2009/0030326 A1
(43) Pub. Date: Jan. 29, 2009

(54) URINARY BLADDER ULTRASONIC DIAGNOSIS APPARATUS AND METHOD OF USE THEREOF

(75) Inventors: Jung-Hoe Kim, Seoul (KR); Seung-Tai Kim, Gyeonggi-do (KR)

Correspondence Address: THE RAFFERTY PATENT LAW FIRM, 1952 Gallows Road, Suite 200, Vienna, VA, 22182-3823, US

(73) Assignee: MCUBE TECHNOLOGY CO., LTD., Seoul (KR)

(21) Appl. No.: 12/045,680

(22) Filed: Mar. 10, 2008

(30) Foreign Application Priority Data
Apr. 25, 2006 (KR) 10-2006-0037132

(51) Int. Cl. A61B 8/14

(52) U.S. Cl. 600/459

(57) ABSTRACT

The present invention relates to an ultrasonic diagnosis apparatus and method for the urinary bladder. The ultrasonic diagnosis apparatus has a preliminary scan mode and a scan mode. The ultrasonic diagnosis apparatus first operates in the preliminary scan mode and operates in the scan mode after accurately detecting the location of the urinary bladder, thus measuring the amount of urine in the urinary bladder. When operating in the preliminary scan mode, the ultrasonic diagnosis apparatus receives pieces of ultrasonic information of n scan lines for a single plane, and acquires and displays an image for a corresponding plane using the pieces of received ultrasonic information. When operating in the scan mode, the ultrasonic diagnosis apparatus sequentially receives pieces of ultrasonic information of n scan lines for each of m planes from a transducer, and calculates the amount of urine in the urinary bladder using the pieces of received ultrasonic information. The ultrasonic diagnosis apparatus according to the present invention operates in the preliminary scan mode, so that the location of the urinary bladder can be quickly and accurately detected, therefore the amount of urine in the urinary bladder also can be quickly and accurately detected.

### TECHNICAL FIELD

The present invention relates generally to a portable ultrasonic diagnosis apparatus for the urinary bladder and an ultrasonic diagnosis method using the apparatus and, more particularly, to a portable and small-sized ultrasonic diagnosis apparatus, which has a preliminary scan mode and a scan mode, thus not only quickly and accurately detecting the location of the urinary bladder but also automatically measuring the amount of urine in the urinary bladder, and an ultrasonic diagnosis method, which can measure the amount of urine in the urinary bladder using the apparatus.

### BACKGROUND ART

Generally, an ultrasonic system is a system that emits ultrasonic signals to an object to be examined using the piezoelectric effect of a transducer, receives the ultrasonic signals reflected from the discontinuous planes of the object, converts the received ultrasonic signals into electrical signals, and outputs the electrical signals to a predetermined display device, thus enabling examination of the internal state of the object. Such an ultrasonic system is widely used for medical diagnosis equipment, non-destructive testing equipment and underwater detection equipment.

However, most conventional ultrasonic diagnosis apparatuses are inconvenient in that they cannot be easily carried due to their large size and heavy weight. To solve the inconvenience, various portable ultrasonic diagnosis apparatuses have been proposed. Korean Utility Model Registration No. 20-137995 discloses a “Portable Ultrasonic Diagnosis Apparatus.”

Meanwhile, when examining bladder abnormalities or urinary difficulty, measuring the amount of urine is an essential procedure. Furthermore, prior to urination using a catheter, the amount of urine in the urinary bladder should be measured to account for urine that may be retained after the operation. In addition, in urination training, the amount of urine in the urinary bladder should be measured as a guideline.

Various types of ultrasonic scanning equipment may be used to measure the amount of urine in the urinary bladder, as described above. In this case, two methods are used. A first method calculates the amount of urine from respective ultrasonic images for a perpendicular plane and a horizontal plane, which are obtained using typical ultrasonic scanning equipment. However, although many algorithms has been proposed and used for the method, the first method is problematic in that it not only exhibits a considerable error rate but also exhibits different results for different users. A second method uses dedicated ultrasonic equipment for measuring the amount of urine. U.S. Pat. No. 4,926,871 discloses dedicated ultrasonic equipment. However, the dedicated ultrasonic equipment based on the second method has a disadvantage in that it also calculates the amount of urine chiefly using two ultrasonic images, which are related to the perpendicular and horizontal planes of the urinary bladder, respectively, and in that a user must find the area indicating the greatest size and select it in order to calculate the amount of urine.

Accordingly, the present applicant proposes a method of accurately calculating the amount of urine in the urinary bladder while minimizing user interference.

### DISCLOSURE

### Technical Problem

In order to solve the above problems, an object of the present invention is to provide an ultrasonic diagnosis apparatus for the urinary bladder, which can not only quickly and accurately detect the location of the urinary bladder but also measure the amount of urine in the urinary bladder.

Another object of the present invention is to provide an ultrasonic diagnosis apparatus for the urinary bladder, which has a size and weight suitable for portable applications.

A further object of the present invention is to provide an ultrasonic diagnosis method in which the ultrasonic diagnosis apparatus thereof can accurately measure the amount of urine in the urinary bladder using received ultrasonic signals.

### Technical Solution

In order to accomplish the above objects, the present invention provides an ultrasonic diagnosis apparatus for a urinary bladder, the ultrasonic diagnosis apparatus measuring the amount of urine in the urinary bladder, the ultrasonic diagnosis apparatus including:

a transducer for emitting ultrasonic signals and receiving ultrasonic signals reflected from an object;

a transducer support configured such that the transducer is fixedly installed therein;

an analog signal processing unit for converting the ultrasonic signals, which are transmitted from the transducer, into digital signals;

a display unit for outputting specific image signals; a central control unit for performing image processing on the digital ultrasonic signals transmitted from the analog signal processing unit, outputting the results of the processing to the display unit, and controlling the overall operation of the apparatus; a first stepping motor for rotating the transducer in a first direction; a second stepping motor for rotating the transducer in a second direction; a drive control unit for controlling the operation of the first and second stepping motors in response to drive control signals provided from the central control unit; and a switch unit for selecting operation modes; wherein, when a first operational mode is selected by the switch unit, the central control unit receives pieces of ultrasonic information of n scan lines for a single plane at a current location from the transducer, acquires an image from the pieces of received ultrasonic information, and outputs the acquired image to the display unit, and when a second operational mode is selected by the switch unit, the central control unit receives pieces of ultrasonic information of n scan lines for each of m planes from the transducer, and calculates the amount of urine in the urinary bladder using the pieces of received ultrasonic information.

In the ultrasonic diagnosis apparatus, when the first operational mode is selected, it is preferred that the central control unit transmit a drive control signal for rotating the second stepping motor at a current location to the drive control unit, the drive control unit sequentially rotate the second stepping motor in response to the drive control signal received from the central control unit, and the central control unit receive the pieces of ultrasonic information of n scan lines, which are transmitted from the transducer, according to the second stepping motor, extract a two-dimensional bladder image for a corresponding plane from the pieces of received ultrasonic information, and output the extracted two-dimensional bladder image to the display unit.

In the ultrasonic diagnosis apparatus, when the second operational mode is selected, it is preferred that the central control unit sequentially rotate the transducer in the first direction by rotating the first stepping motor, and transmit a drive control signal, which is used to rotate the second stepping motor in the second direction by a predetermined angle n times, to the drive control unit whenever the first stepping motor rotates, the drive control unit rotate the first and second stepping motors in response to the drive control signals transmitted from the central control unit, and the central control unit calculate the amount of urine in the urinary bladder using the pieces of ultrasonic information of n scan lines for each of m planes, which are sequentially received from the transducer according to the rotation of the first and second stepping motors.

In addition, the present invention provides an ultrasonic diagnosis method, the ultrasonic diagnosis method measuring the amount of urine in the urinary bladder using an ultrasonic diagnosis apparatus, the ultrasonic diagnosis method including the steps of: (a) determining an operational mode input from an outside; (b) if it is determined that the operational mode input from the outside is a preliminary scan mode, receiving pieces of ultrasonic information of n scan lines for a single plane at a current location from a transducer, extracting a bladder image for a corresponding plane from the pieces of received ultrasonic information, and outputting the extracted image to a display unit; and (c) if it is determined that the operational mode input from the outside is a scan mode, sequentially receiving pieces of ultrasonic information of n scan lines for each of m planes from the transducer, and measuring the amount of urine in the urinary bladder using the pieces of received ultrasonic information.

In the ultrasonic diagnosis method, it is preferred that the step (c) include the steps of: (c1) detecting the locations of front and rear walls from the pieces of ultrasonic information of all of the scan lines; (c2) obtaining difference values between the detected locations of the front and rear walls for the respective scan lines; (c3) obtaining areas for bladder images of the respective planes using the difference values for the scan lines of each plane; (c4) obtaining correction coefficients for the respective planes; (c5) calculating radii of respective circles having areas identical to areas for the bladder images of the respective planes, and calculating corrected radii by applying the correction coefficients for the respective planes to the radii for the respective planes; (c6) obtaining an average radius of the connected radii for the respective planes; and (g) obtaining the volume of a sphere using the average radius. In this case, the finally obtained volume of the sphere is the volume of urine in the urinary bladder.

### Advantageous Effects

According to the present invention, two stepping motors having one transducer and two rotational axes are provided, so that an ultrasonic diagnosis apparatus that not only has small size and weight but also can provide ultrasonic information about a three-dimensional image can be provided.

Furthermore, the two stepping motors of the ultrasonic diagnosis apparatus according to the present invention collect the ultrasonic information while rotating automatically, so that all of the ultrasonic information included in a cone-shaped region from the location at which the ultrasonic diagnosis apparatus is disposed can be collected. As a result, conventional apparatuses measure the amount of urine in the urinary bladder using only ultrasonic information about two planes, and thus data is incorrect, whereas the apparatus according to the present invention measures the amount of urine using ultrasonic information about a plurality of planes that are uniformly spaced throughout 360°, so that it can very accurately measure the amount of urine.

In particular, the apparatus according to the present invention uses correction coefficients that numerically indicate the extent to which the first detected location is displaced from the center of the urinary bladder, so that accurate measurement can be always performed even if the detected location is displaced from the center of the urinary bladder.

Furthermore, the ultrasonic diagnosis apparatus according to the present invention operates in the preliminary scan mode, and thus the central location of the urinary bladder that a user desires to examine can be quickly and accurately detected. As a result, the amount of urine in the urinary bladder can also be quickly and accurately measured.

### DESCRIPTION OF DRAWINGS

FIG. 1 is a block diagram schematically showing the internal construction of an ultrasonic diagnosis apparatus according to a preferred embodiment of the present invention;

FIG. 2 is a perspective view showing the ultrasonic diagnosis apparatus of FIG. 1;

FIG. 3 is a conceptual diagram illustrating a process of acquiring a two-dimensional image using the ultrasonic diagnosis apparatus of FIG. 2; and

FIG. 4 is a flowchart sequentially illustrating a process of obtaining the volume of urine in the urinary bladder using the ultrasonic diagnosis apparatus according to a preferred embodiment of the present invention.

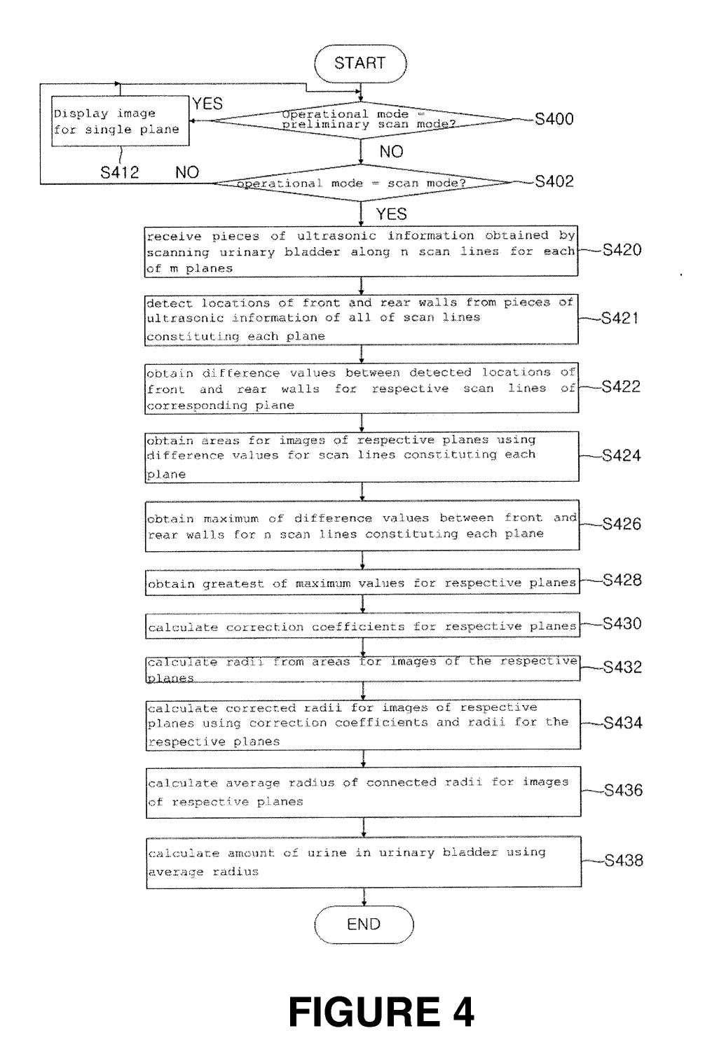

### BEST MODE

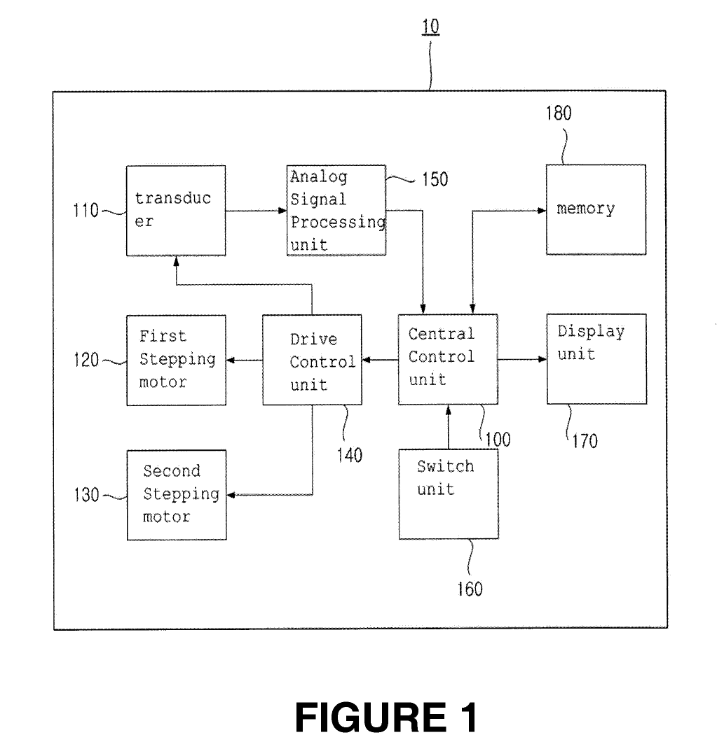

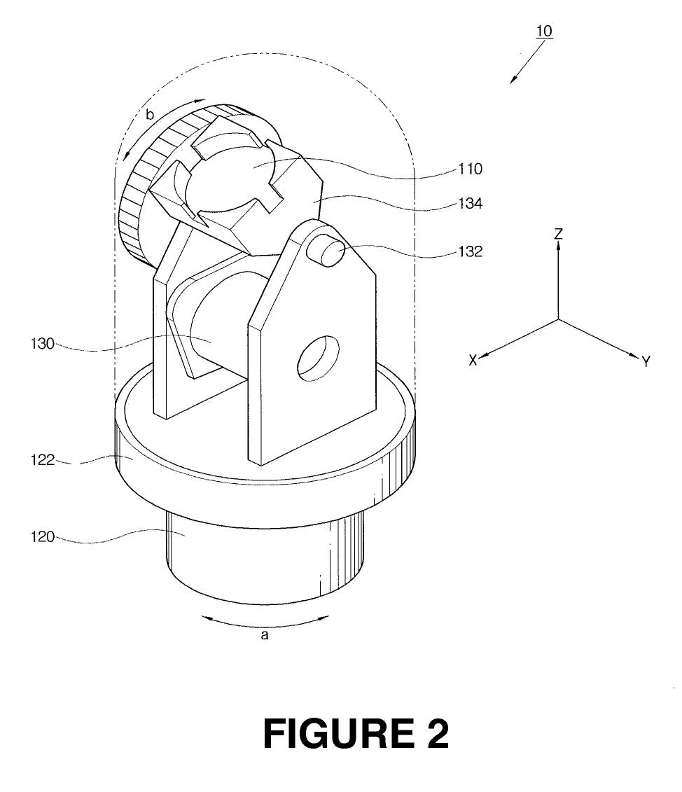

The construction and operation of an ultrasonic diagnosis apparatus for the urinary bladder according to a preferred embodiment of the present invention are described in detail with reference to the accompanying drawings below. FIG. 1 is a block diagram schematically showing the internal construction of an ultrasonic diagnosis apparatus according to the preferred embodiment of the present invention, and FIG. 2 is a perspective view showing the ultrasonic diagnosis apparatus of FIG. 1.

Referring to FIG. 1, the ultrasonic diagnosis apparatus 10 according to the preferred embodiment of the present invention includes a central control unit 100 for controlling the overall operation of the apparatus, a transducer 110, a first stepping motor 120, a second stepping motor 130, a drive control unit 140, an analog signal processing unit 150, a switch unit 160, memory 180, and a display unit 170. The respective components of the above-described ultrasonic diagnosis apparatus 10 are described in detail below.

The transducer 110 is a device that emits ultrasonic signals and receives ultrasonic signals reflected from the internal organs of a human body, and transmits the received analog signals to the analog signal processing unit 150. The transducer 110 of the ultrasonic diagnosis apparatus for the urinary bladder according to the present invention receives ultrasonic signals reflected from urine in the urinary bladder.

The analog signal processing unit 150 converts the analog signals, which are transmitted from the transducer 110, into digital signals, and transmits the digital signals to the central control unit 100.

The switch unit 160 includes a switch for performing input to select operational modes, such as a preliminary scan mode and a scan mode. The switch unit 160 according to a preferred embodiment of the present invention enables an operational mode, depending on input time or input form, to be determined using a single switch. In addition, another embodiment of the switch unit 160 of the present invention may be configured to be provided with a plurality of buttons, and allow different buttons to be assigned to respective operational modes.

The central control unit 100 determines an operational mode based on a signal input through the switch unit. Thereafter, when the preliminary scan mode is determined, an operation is performed in the preliminary scan mode. In contrast, when the scan mode is determined, an operation is performed in the scan mode.

The operation in the preliminary scan mode of the ultrasonic diagnosis apparatus according to the present invention is described below. When the preliminary scan mode is selected, the central control unit transmits a drive control signal for sequentially rotating the second stepping motor to the drive control unit, and the drive control unit rotates the second stepping motor in a yz direction (that is, a second direction) in response to the drive control signal received from the central control unit. As the second stepping motor rotates, the transducer also rotates. The transducer acquires the pieces of ultrasonic information of n scan lines in the yz direction while rotating in the yz direction. Meanwhile, the central control unit receives the pieces of ultrasonic information of n scan lines in the yz direction from the transducer, extracts a bladder image for a corresponding plane in the yz direction from the pieces of received ultrasonic information, and outputs the extracted image to the display unit. In this case, in the state in which the transducer is disposed on the abdomen of a patient and is oriented toward his or her urinary bladder in the preliminary scan mode, the scanning apparatus according to the present invention rotates in left and right directions relative to the patient, that is, a lateral direction with respect to the patient, and thus a two-dimensional image obtained as a result of the rotation is output to the display unit.

A user, who uses the scanning apparatus according to the present invention, causes the scanning apparatus to operate in the preliminary scan mode and then views the image output to the display unit, so that he or she can be quickly and accurately made aware of the location of the urinary bladder which is to be examined.

Furthermore, in the preliminary scan mode, the above-described process is periodically repeated until the scan mode is input and a two-dimensional image for a corresponding plane is output to the display unit. In this case, it is preferred that the repetition period be less than about 5 seconds.

Meanwhile, in another embodiment of the ultrasonic diagnosis apparatus according to the present invention for the preliminary scan mode, when the preliminary scan mode is selected, respective two-dimensional images for three planes are acquired, and are displayed on a single screen. In this case, it is preferred that the acquired three planes for two-dimensional images be formed to have different angles.

The operation of the ultrasonic diagnosis apparatus according to the present invention in the scan mode is described below. When the scan mode is selected, the central control unit 100 rotates the first stepping motor and the second stepping motor, and thus the transducer acquires the pieces of ultrasonic information of n scan lines for each of m planes. A process of the transducer acquiring the pieces of ultrasonic information of n scan lines for each of m planes is as follows.

First, after the first stepping motor is fixed, the transducer acquires the ultrasonic information of a single scan line at a location to which movement is made while the second stepping motor is sequentially rotated n times by a predetermined angle, and thus the pieces of ultrasonic information of n scan lines for a single plane are acquired.

Thereafter, the above-described process (that is, the process of the transducer acquiring the pieces of ultrasonic information of n scan lines for a single plane at the corresponding location) is repeated while the first stepping motor, which moves in a direction orthogonal to the second stepping motor, is sequentially rotated m times by a predetermined angle, and thus the pieces of ultrasonic information of n scan lines for m planes, to which movement is made by the second stepping motor, are acquired.

The first stepping motor and the second stepping motor are rotated as described above, so that ultrasonic waves are emitted and received in the form of a cone, the vertex of which is formed by the transducer, therefore the three-dimensional volume of the urinary bladder can be measured.

Meanwhile, the central control unit 100 receives the pieces of ultrasonic information, which are acquired by the transducer, from the transducer through the analog signal processing unit 150. The central control unit 100 calculates the volume of urine in the urinary bladder, which is an examination object, using the signals transmitted from the analog signal processing unit 150, and outputs the ultrasonic image of the urinary bladder, which is an image related to the specific plane of the urinary bladder, to the display unit 170. The display unit 170 displays the image, which is transmitted from the central control unit, on the screen along with the volume of urine remaining in the urinary bladder.

As shown in FIG. 2, a rotational support 122 is connected to the first stepping motor 120. A second stepping motor 130 is mounted on the rotational support 122 and rotates along with the rotational support 122. The second stepping motor 130 is connected with a transducer support including a rotational axis. A transducer 110 is installed in the transducer support.

The central control unit 100 transmits drive control signals to the drive control unit 140 in response to an operational mode signal received from the switch unit 160, and the drive control unit 140 controls the motion of the first and second stepping motors 120 and 130 in response to the drive control signals, so that the ultrasonic image of the urinary bladder can be captured through the rotation of the transducer 110.

The second stepping motor 130 rotates by the predetermined angle in an yz plane, and the rotational axis 132 and the transducer support 134, which are connected to the second stepping motor via a gear, are rotated by the second stepping motor 130. Consequently, the transducer 110 installed in the transducer support 134 rotates in the second direction (that is, the yz plane).

Meanwhile, the rotational support 122, on which the second stepping motor 130 is mounted, is connected to the first stepping motor 120, so that the rotational support 122 also moves by the predetermined angle in a first direction (that is, an xy direction) as the first stepping motor 120 moves in an xy plane. Accordingly, the second direction, which is the direction in which the second stepping motor rotates, and the first direction, which is the direction in which the first stepping motor rotates, are orthogonal to each other.

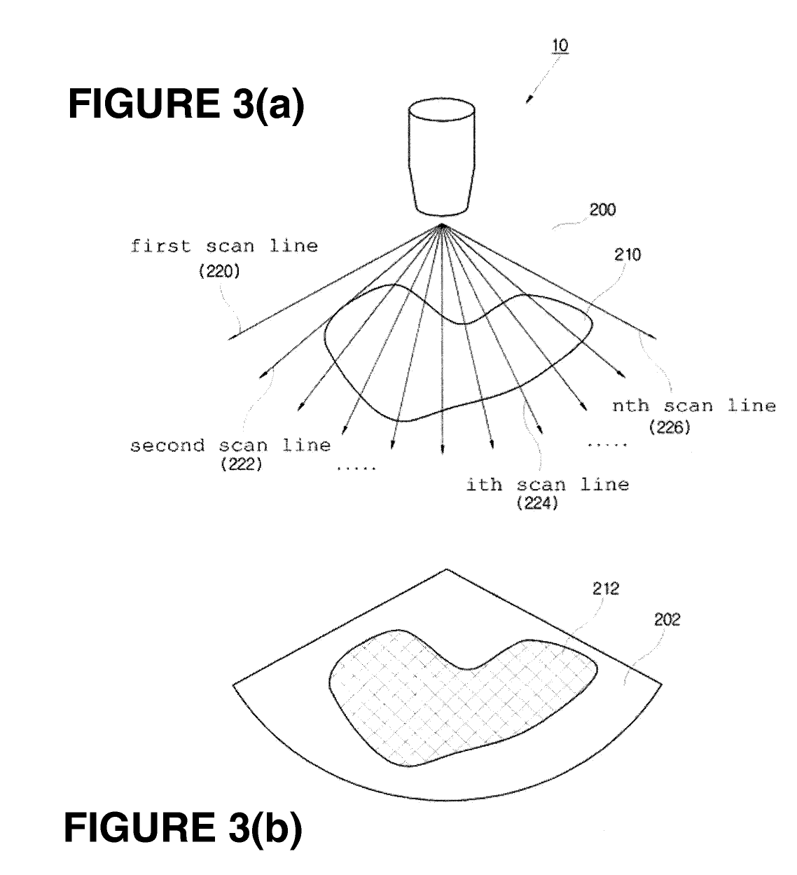

FIGS. 3(a) and 3(b) are diagrams illustrating a process of the ultrasonic diagnosis apparatus 10, according to the present invention, acquiring a bladder image for a single plane. With reference to FIG. 3(a), in the ultrasonic diagnosis apparatus 10 in which the transducer is disposed on an arbitrary location of an abdomen 200 over the urinary bladder 210 of a patient, the central control unit causes the first stepping motor and the second stepping motor to be fixed, and detects ultrasonic signals at the corresponding location. Thereafter, a process of detecting ultrasonic signals at a corresponding angle while moving the second stepping motor by the predetermined angle in the yz direction is repeated, and thus ultrasonic signals for n scan lines, that is, a first scan line 220, a second scan line 222, . . . , ith scan line 224, . . . , nth scan line 226 are sequentially detected. After detecting n ultrasonic signals, the central control unit 100, as shown in FIG. 3(b), generates a two-dimensional image by processing ultrasonic signals for a corresponding plane, and displays the generated two-dimensional image on the display unit 170. FIG. 3(b) is a diagram showing the two-dimensional image output to the display unit 170, in which urine 212 in the urinary bladder 210 is displayed while being separated from organs 202 around the urinary bladder 210.

Meanwhile, the above-described process is repeated while the first stepping motor is rotated by the predetermined angle and, thus, ultrasonic signals for the n scan lines for the m planes are detected. As described above, a three-dimensional image is generated using two-dimensional images acquired for the m planes. In this case, it is preferred that the number m of the acquired two-dimensional images be equal to or greater than 4 and equal to and less than 30.

### Bladder Volume Measurement Method

A method of the central control unit 100 of the ultrasonic diagnosis apparatus 10 according to the preferred embodiment of the present invention, having the above-described construction, measuring the amount of urine in the urinary bladder using ultrasonic signals, is described below.

First, the central control unit determines whether an operational mode, which is input through the switch unit, is the preliminary scan mode or the scan mode at step 400. If it is determined that the operational mode is the preliminary scan mode, pieces of ultrasonic information, which are obtained by scanning n scan lines for a single plane at a current location, are received at step 410. Thereafter, a two-dimensional bladder image for the corresponding plane is extracted from the pieces of received ultrasonic information, and is output to the display unit, at step 412. Accordingly, the user, who manipulates the ultrasonic diagnosis apparatus according to the present invention the present invention, causes the ultrasonic diagnosis apparatus to operate in the preliminary scan mode, and moves a probe or adjusts the tilt angle of the probe while viewing the two-dimensional image displayed on the screen, so that the urinary bladder can be located in the center portion of the ultrasonic image and, in addition, the location and tilt angle of the probe can be detected such that a large bladder plane is viewed. From the above-described process, an operation can be performed in the scan mode at a location close to the center of the urinary bladder, and, as a result, the measurement of the urinary bladder can be accurately and quickly performed.

If it is determined that the operational mode is the scan mode, pieces of ultrasonic information, which are obtained by scanning the urinary bladder, which is an object to be examined, along n scan lines for each of m planes, are received from the transducer of the ultrasonic diagnosis apparatus at step 420. The process of receiving pieces of ultrasonic information of n scan lines for a single plane is repeatedly performed on the m planes, and thus the pieces of ultrasonic information of each of n scan lines for m plans are received. The number of planes to be scanned and the number of scan lines for a single plane may be determined according to the region and size of the object to be examined. In the case of measuring the urinary bladder, the number of scan lines and the number of images may be determined such that the entire region of the urinary bladder can be included. For example, in the case of scanning the urinary bladder, the entire region of the urinary bladder can be sufficiently included using about 67 lines if the angle between lines for forming a single image is 1.8°.

Thereafter, the locations of front and rear walls are detected from pieces of ultrasonic information of scan lines constituting each plane at step S421, and difference values Depth[1], Depth[2], . . . , Depth[n] corresponding to the differences between the locations of the detected front and rear walls for the respective scan lines are obtained at step S422. Thereafter, the area of the corresponding plane is obtained by summing the difference values for the scan lines constituting each plane.

The above-described process of obtaining the area of each plane is repeatedly performed on m planes, and thus the areas Area[1], Area[2], . . . , Area[m] of the respective planes are obtained at step 424. In this case, the method of obtaining the area of each plane using difference values corresponding to the differences between the locations of the front and rear walls of the urinary bladder for the respective scan lines may be implemented in various ways. As an example, the entire area of each plane may be obtained by obtaining an area for a sector for a single scan line using the rotational angle of the second stepping motor 130 and summing sector areas for respective lines having rear walls. As another example, the entire area may be obtained by summing trapezoidal areas, which are obtained by repeating a process of obtaining an area for a trapezoid, which is formed by the two front walls and two rear walls of two neighboring scan lines.

Meanwhile, if scanning is performed in a state in which the center of a first rotational axis moves from the center of the urinary bladder when a three-dimensional volume is obtained using a plurality of two-dimensional images, an amount smaller than an actual amount is calculated and, thus, an error relative to the actual amount is generated. Accordingly, numerical correction is performed to reduce such error and accurately measure the amount of urine in the urinary bladder. The process of performing the numerical correction is described below.

First, difference values corresponding to the differences between the locations of front and rear walls of the urinary bladder for n scan lines constituting each plane are obtained. Thereafter, the maximum difference values bladderDepth[1], bladderDepth[2], . . . , bladderDepth[m] of the respective planes are obtained among the difference values at step 426, and the greatest ‘MaxbladderDepth’ of the maximum difference values of the respective planes is obtained at step S428.

Thereafter, at step 430, the correction coefficients ComFactor[1], ComFactor[2], . . . , ComFactor[i], and ComFactor[m] for the respective planes are obtained using the greatest ‘MaxBladderDepth’ of the maximum difference values and the maximum difference values BladderDepth[1], BladderDepth[2], . . . , BladderDepth[m] of the respective planes, based on the following Equation 1.

$$ComFactor [ i ] = \frac{Max BladderDepth}{BladderDepth [ i ]} \qquad [ Equation 1 ]$$

Thereafter, given the assumption that a bladder image for each plane is a circle, radii r[1], r[2], . . . , r[i], and r[m] of respective circles having the same areas as the areas Area[1], Area[2], . . . , Area[m] of the respective planes are obtained and are determined to be radii for bladder images of the respective planes at step S432.

Thereafter, at step S434, corrected radii ComR[1], ComR[2], . . . , ComR[i], and ComR[m] with respect to the correction coefficients and the radii for the urinary bladder images of the respective planes are obtained using the following

ComR[i]=ComFactor[i]×r[i] [Equation 2]

An average radius ‘AverageR’, which is the average value of the calculated corrected radii for the images of the respective planes, is obtained at step S436. Thereafter, given the assumption that the complete bladder is a sphere, the total volume V of urine in the urinary bladder is obtained by applying the average radius to the following Equation 3 at step S438.

$$V = \frac{4}{3} π {AverageR}^{3} \qquad [ Equation 3 ]$$

From the above-described process, the ultrasonic diagnosis apparatus for the urinary bladder according to the present invention can accurately detect the amount of urine in the urinary bladder.

Furthermore, the ultrasonic diagnosis apparatus for the urinary bladder according to the present invention can extract pieces of bladder information, such as the thickness and weight of the urinary bladder, as well as information about the amount of urine remaining in the urinary bladder, from two-dimensional images, and can output the pieces of extracted information of the urinary bladder to the display unit.

Although the present invention has been described in detail in conjunction with the preferred embodiment, the present invention is described only for illustrative purposes, and is not limited thereto. Those skilled in the art will appreciate that various modifications and applications, which are not described above, are possible within a range that does not change the substantial characteristics of the present invention. For example, in the present embodiment, the method of obtaining the area of a corresponding plane using the rotational angles of the first stepping motor and the second stepping motor and ultrasonic information about the respective scan lines may be modified and implemented in various ways to improve scanning performance. Furthermore, it should be appreciated that the differences regarding the modifications and the applications are included in the scope of the present invention, which is defined by the accompanying claims.

### INDUSTRIAL APPLICABILITY

The ultrasonic diagnosis apparatus and method according to the present invention may be widely used in the medical field.

1. An ultrasonic diagnosis apparatus for a urinary bladder, comprising:
a transducer for emitting ultrasonic signals and receiving ultrasonic signals reflected from an object;
a transducer support configured such that the transducer is fixedly installed therein;
an analog signal processing unit for converting the ultrasonic signals, which are transmitted from the transducer, into digital signals;
a display unit for outputting specific image signals;
a central control unit for performing image processing on the digital ultrasonic signals transmitted from the analog signal processing unit, outputting results of the processing to the display unit, and controlling overall operation of the apparatus;
a first stepping motor for rotating the transducer in a first direction;
a second stepping motor for rotating the transducer in a second direction;
a drive control unit for controlling operation of the first and second stepping motors in response to drive control signals provided from the central control unit; and
a switch unit for selecting operation modes;
wherein, when a first operational mode is selected by the switch unit, the central control unit receives pieces of ultrasonic information of n scan lines for a single plane at a current location from the transducer, acquires an image from the pieces of received ultrasonic information, and outputs the acquired image to the display unit, and
when a second operational mode is selected by the switch unit, the central control unit receives pieces of ultrasonic information of n scan lines for each of m planes from the transducer, and calculates an amount of urine in the urinary bladder using the pieces of received ultrasonic information.

2. The ultrasonic diagnosis apparatus according to claim 1, wherein, when the first operational mode is selected, the central control unit transmits a drive control signal for rotating the second stepping motor at a current location to the drive control unit,
the drive control unit sequentially rotates the second stepping motor in response to the drive control signal received from the central control unit, and
the central control unit receives the pieces of ultrasonic information of n scan lines, which are transmitted from the transducer, according to the second stepping motor, extracts a two-dimensional bladder image for a corresponding plane from the pieces of received ultrasonic information, and outputs the extracted two-dimensional bladder image to the display unit.

3. The ultrasonic diagnosis apparatus according to claim 1, wherein, when the second operational mode is selected, the central control unit fixes the first stepping motor and acquires ultrasonic information while sequentially rotating the second stepping motor n times by a predetermined angle, thus acquiring the pieces of ultrasonic information of n scan lines for a single plane, and
the central control unit acquires the pieces of ultrasonic information of n scan lines for each of m planes by repeating the above-described process (that is, the process of acquiring the pieces of ultrasonic information of n scan lines for a single plane) m times while sequentially rotating the first stepping motor by the predetermined angle.

4. The ultrasonic diagnosis apparatus according to claim 3, wherein the central control unit detects locations of front and rear walls of the urinary bladder for the respective scan lines, obtains difference values corresponding to differences between the detected locations of the front and rear walls for the respective scan lines, obtains areas for bladder images of the respective planes using the difference values for n scan lines constituting each plane, obtains correction coefficients for the respective planes, calculates radii of respective circles having areas identical to the areas for the urinary bladder images of the respective planes, and calculating corrected radii for the respective planes by applying the correction coefficients to the calculated radii for the respective planes, obtains an average radius of the connected radii for the respective planes, and obtains a volume of a sphere using the average radius.

5. The ultrasonic diagnosis apparatus according to claim 4, wherein the central control unit detects a maximum of the difference values for the respective scan lines for each plane, obtains a greatest of the maximum values for the respective planes, and obtains the correction coefficients for the respective planes using ratios of the maximum values for the respective planes to the greatest of the maximum values.

6. An ultrasonic diagnosis method, the ultrasonic diagnosis method measuring information about a bladder using an ultrasonic diagnosis apparatus, comprising the steps of:
(a) determining an operational mode input from an outside;
(b) if it is determined that the operational mode input from the outside is a preliminary scan mode, receiving pieces of ultrasonic information of n scan lines for a single plane at a current location from a transducer, extracting a bladder image for a corresponding plane from the pieces of received ultrasonic information, and outputting the extracted image to a display unit; and
(c) if it is determined that the operational mode input from the outside is a scan mode, sequentially receiving pieces of ultrasonic information of n scan lines for each of m planes from the transducer, and detecting the urinary bladder information using the pieces of received ultrasonic information.

7. The ultrasonic diagnosis method according to claim 6, wherein the step (c) comprises the steps of:
(c1) detecting locations of front and rear walls of the urinary bladder for the respective scan lines;
(c2) obtaining difference values between the detected locations of the front and rear walls for the respective scan lines;
(c3) obtaining areas for bladder images of the respective planes using the difference values for the scan lines of each plane;
(c4) obtaining correction coefficients for the respective planes;
(c5) calculating radii of respective circles having areas identical to areas for bladder images of the respective planes, and calculating corrected radii by applying the correction coefficients for the respective planes to the radii for the respective planes;
(c6) obtaining an average radius of the connected radii for the respective planes; and
(g) obtaining a volume of a sphere using the average radius,
wherein the ultrasonic diagnosis method measures an amount of urine remaining in the urinary bladder.

8. The ultrasonic diagnosis method according to claim 7, wherein the step (c4) comprises the steps of:
(1) detecting a maximum of the difference values for the respective scan lines for each plane;
(2) obtaining a greatest of the maximum values for the respective planes; and
(3) obtaining correction coefficients for the respective planes using ratios of the maximum values for the respective planes to the greatest of the maximum values.

9. The ultrasonic diagnosis method according to claim 8, wherein the corrected coefficients of the step (3) are calculated using the following Equation 4:
$$ComFactor [ i ] = \frac{Max BladderDepth}{BladderDepth [ i ]} \qquad [ Equation 4 ]$$
where ComFactor[i] is a corrected coefficient for an ith plane, bladderDepth[i] is a maximum of the difference values between locations of front and rear walls for scan lines for the ith plane, and MaxBladderDepth is a greatest of maximum values of the respective planes.

10. The ultrasonic diagnosis method according to claim 6, wherein the urinary bladder information detected by the information the ultrasonic diagnosis method includes at least one of an amount of urine in the urinary bladder, a thickness of the urinary bladder, and a weight of the urinary bladder.

11. An ultrasonic diagnosis method, the ultrasonic diagnosis method detecting information about a bladder using an ultrasonic signal, comprising the steps of:
(a) determining an operational mode input from an outside;
(b) if it is determined that the operational mode input from the outside is a preliminary scan mode, receiving pieces of ultrasonic information of n scan lines for at least one plane at a current location from a transducer, extracting a two-dimensional bladder image for a corresponding plane from the pieces of received ultrasonic information, and outputting the extracted two-dimensional image to a display unit; and
(c) if it is determined that the operational mode input from the outside is a scan mode, sequentially receiving pieces of ultrasonic information of n scan lines for each of m planes from the transducer, extracting m two-dimensional images from the pieces of received ultrasonic information, detecting the urinary bladder information from the extracted m two-dimensional images, and outputting the detected bladder information to the display unit,
wherein the step (b) is periodically performed at regular intervals until the scan mode is selected as the operational mode input from the outside.

12. The ultrasonic diagnosis method according to claim 11, wherein, at the step (c), the urinary bladder information output to the display unit includes at least one of an amount of urine in the urinary bladder, a thickness of the urinary bladder, and a weight of the urinary bladder.

13. The ultrasonic diagnosis method according to any of claims 6 to 11, wherein the number m of the acquired two-dimensional images is equal to or greater than 4 and equal to and less than 30.

14. The ultrasonic diagnosis method according to claim 11, wherein the repetition period of the step (b) is less than 5 seconds.

15. The ultrasonic diagnosis method according to claim 6, wherein, at the step (b), the two-dimensional image detected in the preliminary scan mode is a lateral image acquired through scanning in a lateral direction of a patient using the transducer direction.

16. The ultrasonic diagnosis method according to claim 6, wherein the preliminary scan mode of the step (b) allows two-dimensional images for a maximum of three planes to be acquired, and allows the acquired images to be displayed on a single screen.
